(19) United States
(12) Patent Application Publication
(10) Pub. No.: US 2015/0202073 A1
(43) Pub. Date: Jul. 23, 2015

(54) HEAD AND CERVICAL SPINE POSITION ARTICULATING DEVICE

(71) Applicant: AKTINA CORP., Congers, NY (US)

(72) Inventors: Nicholas G. Zacharopoulos, Congers, NY (US); Milan Markovic, Wayne, NJ (US)

(73) Assignee: AKTINA CORP., Congers, NY (US)

(21) Appl. No.: 14/602,168

(22) Filed: Jan. 21, 2015

Related U.S. Application Data
(60) Provisional application No. 61/929,647, filed on Jan. 21, 2014.

(51) Int. Cl. A61F 5/37

(52) U.S. Cl. CPC A61F 5/3707

(57) ABSTRACT

An articulating device to position the head and spine during imaging and treatment of a patient. The device provides mechanism to lock a patient's head using a posterior head support, and anterior face mask or a nose depressor. The device is attached to a patient couch and allows for controlled movement of the head while monitoring and capturing position to provide for returning to the same position in future imaging or treatment. The device includes pitch, roll and translation mechanisms including adjustment controls as well as sensors to capture a patient's position. The device includes sensors which can feed into a controller for automated to remote control of the movement mechanisms if motors or hydraulic controllers are used.

### CROSS-REFERENCE TO RELATED APPLICATION

This non-provisional application claims priority from pending provisional Application No. 61/929,647 filed in the United States Patent and Trademark Office on Jan. 21, 2014.

### BACKGROUND

A Radiation Oncology Linear accelerator is used to deliver a focused radiation beam to the patient. The patient is commonly located on the patient's couch. The treatment plan defines duration of the treatment and it may vary from single delivery to the multiple daily fractionated treatments spread over extended length of time. Factors that influence patient's treatment include accurate patient positioning and location of the treated area, repositioning of the patient for consecutive fractionated treatments, immobilization of the patient during the treatment and patient comfort.

Positioning and immobilizing of the patient undergoing radiation therapy for head and neck or cervical spine is commonly done by use of thermoplastic mask and sheets. The softened thermoplastic sheet is placed over patient's area of interest and attached to the device placed on the patient's couch. Once it hardens, the sheet takes the shape of the patient's body. The patient can be then placed into the same position relative to the patient's couch. There are several disadvantages for this existing approach which this invention resolves along with other advantages of this invention:
Position of the patient's anatomy depends on the initial patient's position during formation of the thermoplastic sheet. If the position is not optimal for the treatment it may reduce effectiveness of the treatment or require repeated formation of thermoplastic sheet.
Patient position cannot be manipulated in order to give maximum exposure of the area of interest. Patient's head cannot be rotated or neck extended or contracted after initial positioning.
It is common that the area of interest is designated using tattoo marks on the patient. The thermoplastic sheet obscures a patient's anatomy thus inhibiting visual location of the area of interest on the patient's body.
For extended treatments, patient's body mass may change thus providing insufficient immobilization (loose thermoplastic if patient loses weight) or making the immobilization device uncomfortable (if patient gain weight or swells during the treatment).
Devices used for immobilization increase attenuation of the beam and may cause adverse effects on patient's health (damage of the skin)
Some of the patients cannot tolerate thermoplastic masks that cover patient's face and/or upper torso (claustrophobic patients).
Setup procedure may be complicated and it may take long time to reproduce.

### BRIEF DESCRIPTION OF THE FIGURES

FIG. 1 depicts the head and cervical spine position articulating device attached to the treatment couch.

FIG. 2 depicts major components of the head and cervical spine position articulating device when used with head mask.

FIG. 3 depicts major components of the head and cervical spine position articulating device when used with nasal depressor.

FIG. 4 depicts subassemblies of the head and cervical spine position articulating device if used with anterior thermoplastic face mask.

FIG. 5 depicts subassemblies of the nasal depressor system.

FIG. 6 depicts major components of the nasal depressor attachment assembly.

FIG. 7 depicts major components of the head support and the mechanism for pitch rotation motion.

FIG. 8 depicts major components of the roll rotation motion mechanism.

FIG. 9 depicts major components of the mechanism for liner superior/inferior movement.

FIG. 10 depicts 6 possible movements of the patients head (3 rotations and 3 translations).

### PART NUMBERS

1 Base plate
2 Bushing, (in one embodiment: 0.375×0.50×25 lg; Flanged; SAE 841)
3 Thrust bearing, (in one embodiment: 0.375″)
4 Knob, (in one embodiment: ⅜×2.5 OD)
5 Threaded collar (in one embodiment: ⅜-16; two piece; SS)
6 Pull handle, (in one embodiment: 3.5′ C-to-C, aluminum, blk, 10-32)
7 Collar cover
8 Spindle, x axis
9 Aluminum rod modified
10 Label kit x axis
11 Superior end plate
12 Inferior end plate
15 Indexing peg
17 Superior travel plate
18 Roll spindle support
19 Roll spindle idle support
20 Roll spindle
21 Nut
22 Knob spacer (in one embodiment: ⅜″)
23 Shaft
24 Washer, (in one embodiment: 0.75×1.50×0.062; PTFE)
25 Pin, phenolic; (in one embodiment: 0.1875×0.75″lg)
26 Head support
27 Pitch spindle bracket
28 Head support bracket LH
29 Head support bracket RH
30 Shaft long
31 Spindle, pitch
32 Mask locating pin
34 Mask lock base
35 Mask lock retainer
36 Locking tab, bottom
37 O-ring
38 Top lock
39 Lock pin
41 Thermoplastic mask frame
42 Universal link
43 Barrel nut slider
44 Dual axis tilt gauge
45 Block support
46 Barrel connector
47 Cam Lever
48 Threaded stud
49 Locknut (in one embodiment: ¼″-20× 7/16 W× 13/64 Ht W/Nylon Insert, SS 18-8)
50 Pin dowel (in one embodiment: 0.187×0.625; SS 18-8)
51 Coiled pin (in one embodiment: 3/32×0.625; High strength, SS420)
53 Adapter base
54 Gauge seat
55 Alignment pin, block
57 Ball seat
58 Ball retainer
59 Ball, split, Nylon
61 Cam seat, split
62 Shimming washer, (in one embodiment: 0.25×0.375×0.060 SS18-8)
100 Head and cervical spine position articulating device shown with thermoplastic face mask
101 Patient couch
102 Pitch and roll angle transducer with display
103 Pitch adjustment knob
104 Translation adjustment knob
105 Translation position indicator scale
106 Roll adjustment knob
107 Patients posterior thermoplastic head support
108 Patients thermoplastic face mask
109 Mask locking mechanism
110 Couch adapter platform with locking mechanism
111 Nose depressor adapter
112 Nose depressor
113 Head support with pitch adjustment mechanism
114 Traveling box with roll adjustment mechanism
115 Device base plate with translation adjustment mechanism
116 Couch locking mechanism
117 Articulating Adapter Block
118 Articulating Nose depressor support assembly

### SUMMARY

The head and cervical spine position articulating device is intended to be used for imaging and treatment of the head and neck and/or cervical spine area. This area includes anatomy inferior to orbits (eye socket openings). Inferior limits of the treatment area include at least complete cervical spine C1 to C7. The cervical spine is difficult to immobilize and/or reposition the patients for multiple treatments. By utilizing head and cervical spine position articulating device the position of the head and cervical spine can be manipulated in order to produce desired patient's posture: The neck can be hyperextended, the head can be rotated left or right or up and down. The patient's body may be immobilized using shoulder restraints that are outside of the treatment field. After immobilizing patient's head within the head and cervical spine position articulating device (using the posterior head support and anterior thermoplastic face mask or nasal depression) the position of the head and cervical spine can be controllably manipulated for initial patient setup or due to required changes in the treatment. The head and cervical spine position articulating device does not have any components in the treatment area thus eliminate any attenuation or artifacts in the treated area and improve effectiveness of the treatment.

Some of the main features of the head and cervical spine position articulating device are:

A. The mechanism of the head and cervical spine position articulating device is position superior to the patient's head in order not to interfere with the area of treatment.

B. When head and cervical spine position articulating device is used there are no components or immobilization devices in the treatment area thus eliminating increased attenuation and simplifying treatment planning.

C. Rotational axis for head roll and pitch is located close to natural or desired axis of rotation.

D. The head and cervical spine position articulating device as designed permits 3 degrees of freedom for patient's head positioning: Two rotations pitch and roll (providing flexing of the neck in around two axis) and one translation (extending and retracting the neck region). While these 3 degrees are most useful in initial patients positioning the device can be equipped with additional two translation and one rotation assuring complete 6 degrees of freedom.

E. The head and cervical spine position articulating device can be manipulated at any time permitting changes in the treatment without need for creating new immobilization component.

F. All of the positions of the head and cervical spine position articulating device are accurately and uniquely reproducible due to the built-in sensors including use of scales and transducers as warranted.

G. The posterior head support is custom molded for each patient assuring comfortable and reproducible positioning. The patient specific head support also assures quick repositioning since it does not require specific adjustment for patient's anatomy. Upon finishing of the complete course of the treatment the head support is disposed.

H. Patient's Anterior thermoplastic mask assures complete immobilization of the patient's head and mandibulae. Simple and reproducible immobilization of mandibulae is crucial for some of the radiation treatment procedures. Anterior thermoplastic mask can be customized with optional openings for patient's eyes and/or eyes and mouth for specific cases (for example for claustrophobic patients). Upon finishing of the complete course of the treatment anterior thermoplastic mask is disposed.

I. If the anterior mask is not desirable, the movement of the patient's head within the device can be restricted using nasal depression. This device is adjusted and customized for the patients in beginning of the treatment and does not require adjustment for multiple treatments. This reduces the treatment preparation time and patient's discomfort. Only the nasal depressor with customizable cushion comes in contact with the patient's face, and this is only disposable parts. All other components of the nasal depression system are reusable upon completion of the treatment.

### DETAILED DESCRIPTION

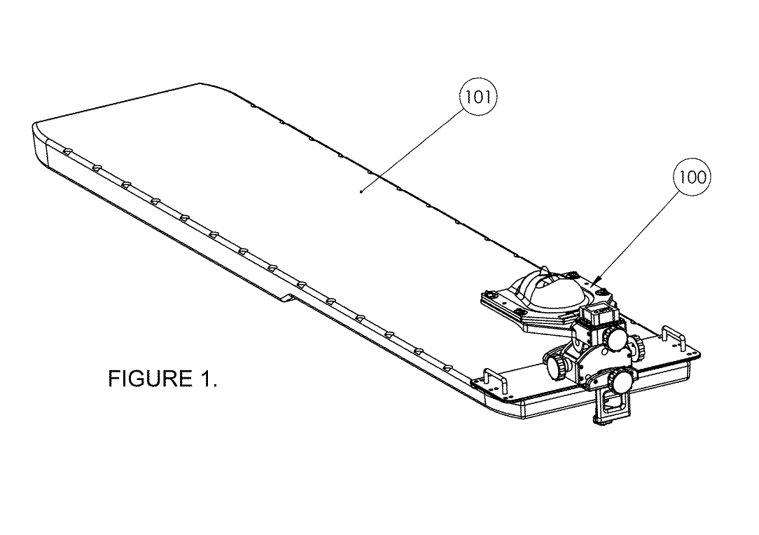

As depicted on FIG. 1 shows the head and cervical spine position articulating device is attached to the superior end of the patients couch (102). The device permits controlled movement of the patient's head. The patient can placed on the couch in prone or supine position. The body of the patient may be restrained in order to minimize the movement. Common devices used for restraining are various types of shoulder and arm restraints.

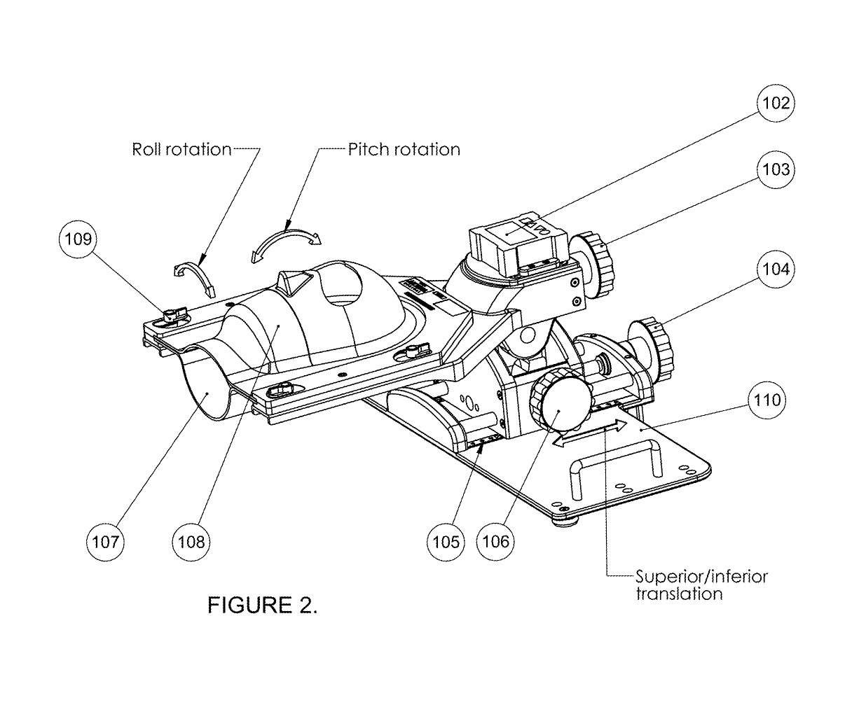

FIG. 2 shows the device setup for the patients in supine position. The posterior head support (107) and anterior face mask (108) are formed prior to the treatment. The patient's head is placed into custom fitted posterior head hammock (107). Once a patient's head is comfortably placed into the device the face mask (108) is secured using mask locking mechanism (109). The relative position of the head in reference to stationary patient's′ body may be manipulated as follows. The head can be rolled sided to side by rotating knobs (106) (one knob on each side of the device for easy access). Pitch of the head can be manipulated by rotating knob (103). The amount of angular displacement from starting position can be recorded or monitored by dual axis angular transducer (102). The transducer has display and/or signal outputs. The movement of the head in superior/inferior direction (stretching or compressing the patient's neck) can be controlled by rotating knob (104). Translation position indicator scale (105) can be used for monitoring and recording superior/inferior position of the patient's head. The scale may be replaced with linear transducer in order to electronically control the position.

The outputs from the dual axis angular transducer (102) and linear transducer for superior/inferior position (not shown) can be used to control the motion in case that device is motorized. For example numerically controlled motors or hydraulically controlled manipulators can replace manual knobs 103,104 and 106). Any controllable movement devices can also be used. And the controllable movement devices can feed into a controller to automate or remotely control these controllable movement devices. Cameras which can, if desired, be used and calibrated for determining exact positions can also be included in an automated or remote controlled set up for the device.

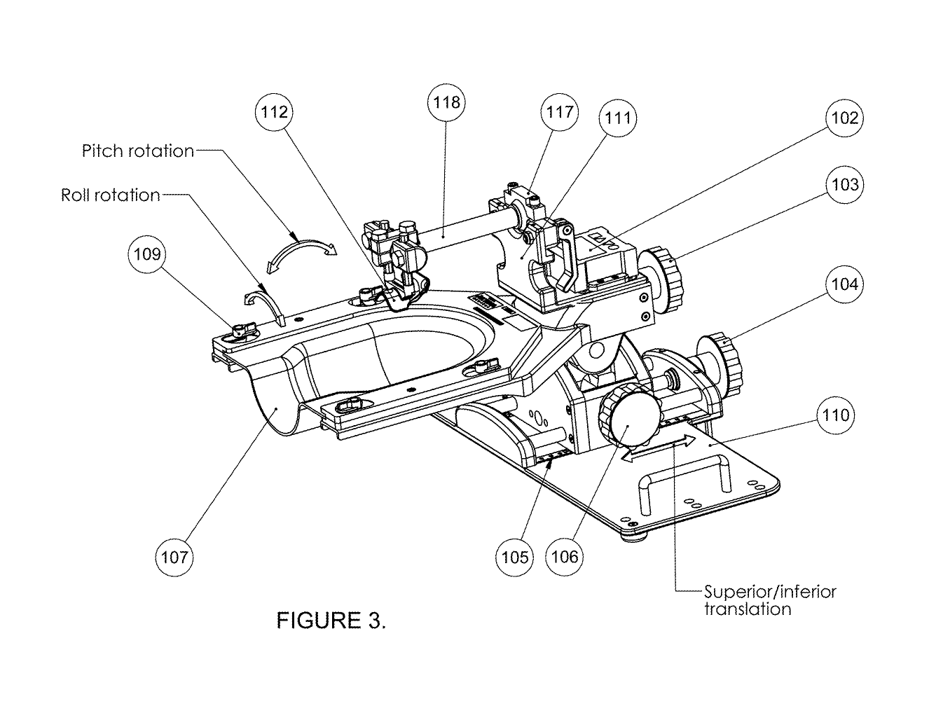

If a face mask is not required or desired (for example claustrophobic patients) immobilization of the patient's head may be achieved using nasal depression. The device as shown on FIG. 3 is equipped with Nose depressor adapter (111). The posterior head support (107) is formed prior to the treatment. The patient's head is placed into custom fitted posterior head hammock (107). Once patient's head is comfortably placed into the device articulating adapter block (117) and nose depressor support assembly (118) are attached to the device. The custom nose depressor (112) is placed onto the patient nasal. The articulating joints of the adapter block (117) and depressor support assembly (118) are secured to the nose depressor (112). Upon finishing initial treatment the whole assembly (consisting of 117,118 and 112) is removed from the nose depressor adapter (111). The assembly is reused without adjustment for consecutive treatments.

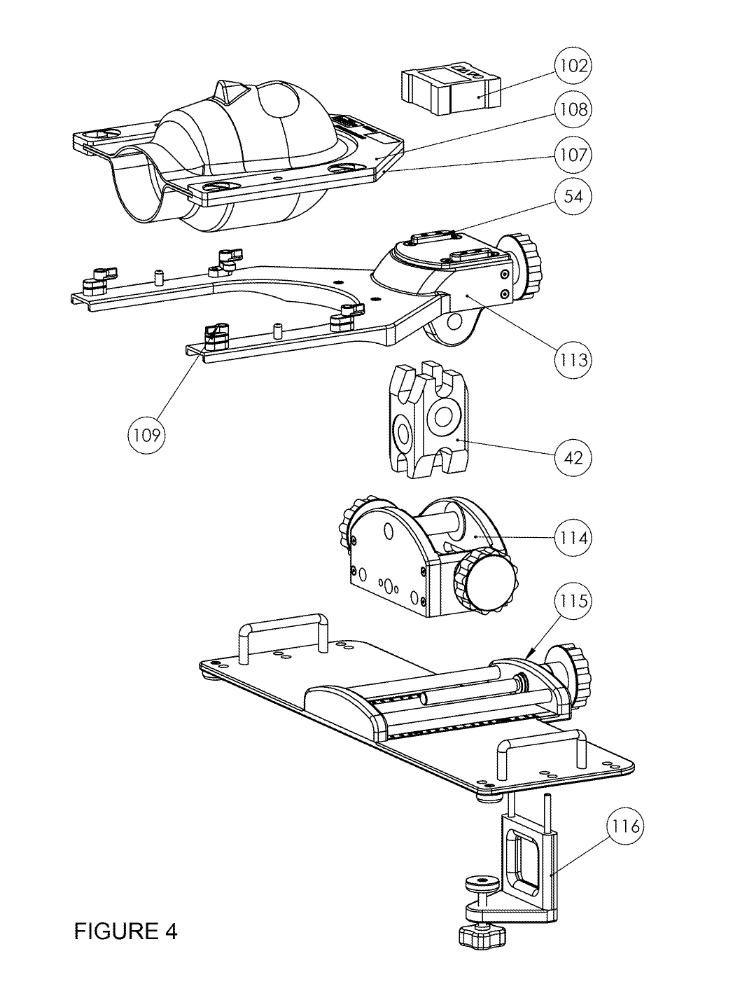

FIG. 4 shows main functional components of the device when used with anterior thermoplastic mask. Patient's posterior thermoplastic head support (107) and patient's thermoplastic face mask (108) are attached to the head support with Pitch adjustment mechanism (113) by Mask locking mechanism (109). The head support with Pitch adjustment mechanism (113) also contain designated docking seat (54) for the Pitch and roll angle transducer with display (102). The whole head support with Pitch adjustment mechanism (113) is connected to the Traveling box with roll adjustment mechanism (114) by Universal link (42). The universal link 42 permits two rotations (pitch and yaw as described in FIG. 2) of the head support (113). Traveling box moves in superior/inferior directions by sliding on linear rails of the Device base plate with translation adjustment mechanism (115). The complete device is secured to the patient's couch (as shown in FIG. 1) by Couch locking mechanism (116). The Couch locking mechanism (116) varies in the design depending of the specific couch with well know methods know to those involved with these tables and their use.

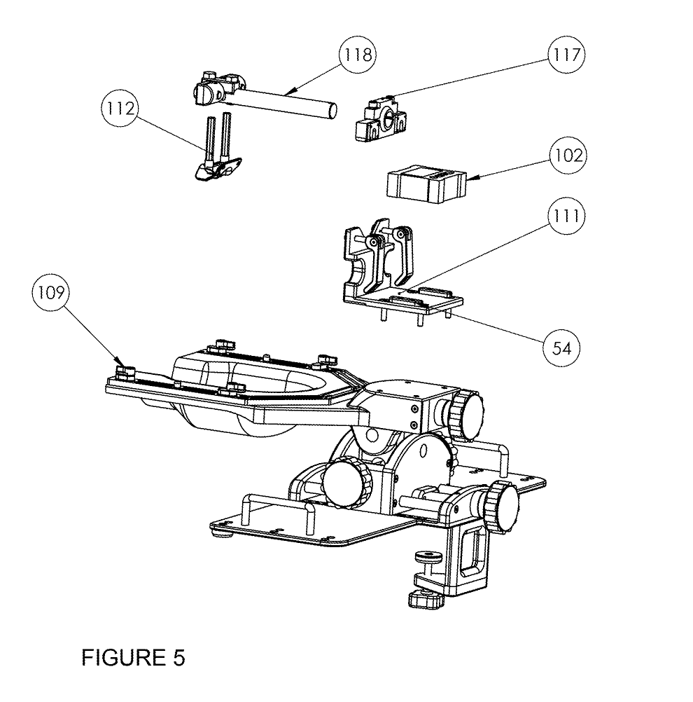

FIG. 5 shows main functional components of the device when used with nasal depression assembly. The device is equipped with nose depressor adapter (111) that permits attachment of the pitch and roll angle transducer with display (102) into the docking seat (54). The nose depressor adapter (111) also assures locking of the articulating adapter block (117). The articulating adapter block (117) includes a spherical joint that permits rotational and linear (superior/inferior) adjustment of the position of the articulating nose depressor support assembly (118). The articulating nose depressor support assembly (118) has built-in attachment for the nose depressor (112) that allows rotational and linear adjustment. The nose depressor (112) is equipped with patient-specific liner for patient's comfort.

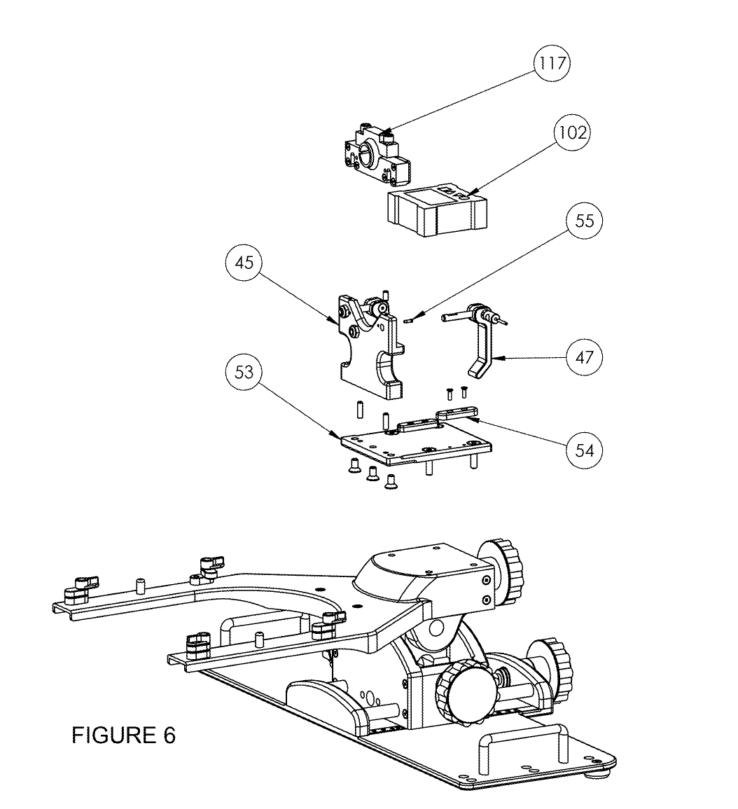

FIG. 6 shows main components of nose depressor adapter (111). The adapter base (53) connects block support (45) to the head support with pitch adjustment mechanism (113). The Articulating Adapter block (117) is located to the block support (45) by alignment pins (55) and secured by cam levers (47). Pitch and roll angle transducer with display (102) is located on the adapter base (53) by docking seat (54).

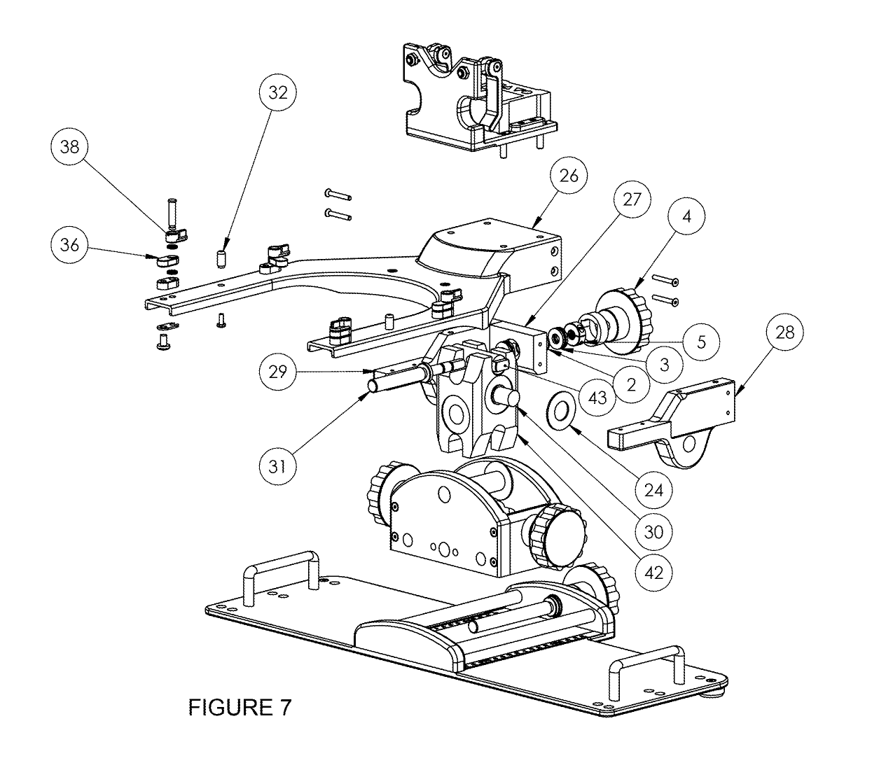

FIG. 7 Shows main components of head support assembly with Pitch adjustment mechanism. The head support (26) is attached to the head support brackets (28 and 29) and the pitch spindle bracket (27). The pitch spindle (31) can freely rotate within the pitch spindle bracket (27) The bushing (2) and thrust bearings (3) assure smooth and accurate movement. The backlash of the system is minimized by securing axil movement with threaded collar (5). The spindle (31) is engaged with Barrel nut slider (43). Rotation of the knob (4) induces rotation of the spindle (31) and causes rotational movement (pitch) of the head support (26) around fixed shaft (30). Mask locating pin (32) assures precise location of the posterior head support (107 and anterior mask 108 (as seen in FIG. 2). Mask locking mechanism has two stages depending if only posterior head support (107) or both posterior head support (107 and anterior mask (108) are used (as seen in FIG. 2). On FIG. 7 the locking tab bottom (36) is used to lock posterior head support (107) only. If both posterior head support (107) and anterior mask (108) are used the locking tab (38) secures the assembly.

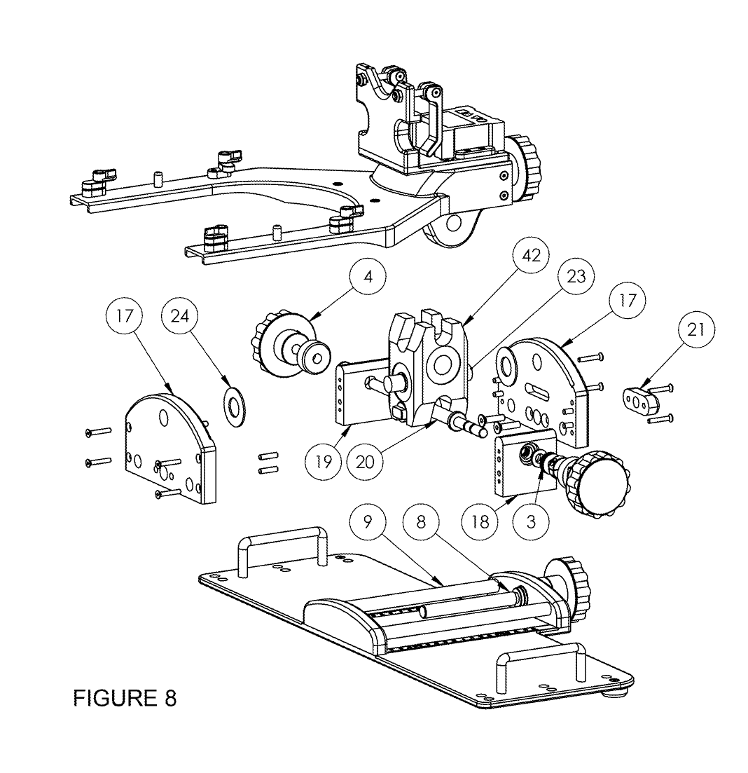

FIG. 8 shows the main components the roll adjustment mechanism as well as the components of the linear superior/inferior movement. Rotation in roll direction (as shown on FIG. 2) is achieved by rotating universal link (42) around fixed shaft (23). The spindle supports (18 and 19) secure spindle (20). The roll spindle (20) can freely rotate within the box formed by travel plates (17) and spindle brackets (18 and 19). The bushing (2) and thrust bearings (3) assure smooth and accurate movement. The backlash of the system is minimized by securing axil movement with threaded collar (5). The spindle (20) is engaged with barrel nut slider (43). Rotating the knob (4) causes the spindle 20) to push barrel nut (43) and cause rotation of the universal link (42). The nut (21) is secured to the travel plate (17). The travel plates (17) are sliding over linear rails (9).

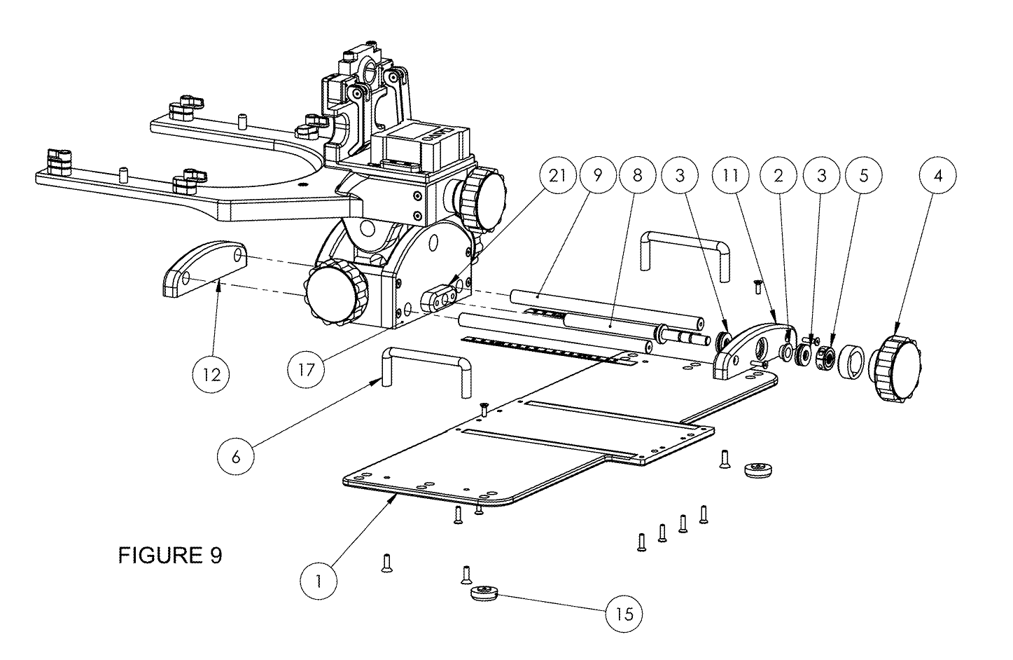

FIG. 9 shows the main components of the attachment base with the mechanism for linear superior/inferior movement. The base plate (1) with handles (6) and indexing pegs (15) are used to position whole device onto the patients couch (as seen in FIG. 1). The base plate (1) on FIG. 9 also has attached superior and inferior end plates (12 and 11). The end plates (12 and 11) support linear rails (9). The spindle (8) used for superior/inferior movement is attached to the superior end plate (11). The bushing (2) and thrust bearings (3) assure smooth and accurate movement. The backlash of the system is minimized by securing axial movement with threaded collar (5). The linear rails (9) assure accurate movement of the travel plates (17). The movement is facilitated by rotation of the knob (4) that causes rotation of the spindle (8) and movement of the engaged nut (21).

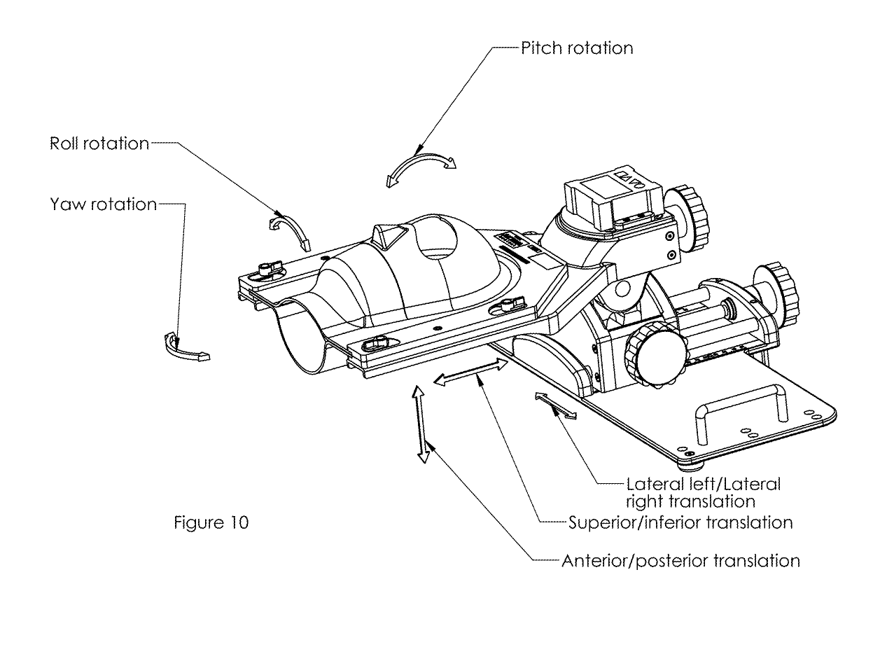

FIG. 10 shows all possible adjustments of the patients head in regard to the patients couch (as described in FIG. 1). Pitch of the head is defined as a rotation of the head in sagittal plane, roll is rotation of the head in transverse plane and yaw is rotation of the head in coronal plane. The Head and cervical spine position articulating device described in all figures incorporates only two rotations and 1 translation (as described in FIG. 2). The other two translation and one rotation can be incorporated as needed: Refer to FIG. 9: Dual axis stage (lateral left/right and Superior inferior direction) and rotary stage (yaw motion) can be added between base plate 1 and Superior and inferior endplates (11 and 12 respectively). These 3 additional movements will accomplish all positioning of the patients head at any position within the range of the movement of the Head and cervical spine position articulating device.

The above is a detailed description of particular embodiments of the invention. It is recognized that departures from the disclosed embodiments may be made within the scope of the invention and that obvious modifications will occur to a person skilled in the art. Those of skill in the art should, in light of the present disclosure, appreciate that many changes can be made in the specific embodiments which are disclosed herein and still obtain a like or similar result without departing from the spirit and scope of the invention. All of the embodiments disclosed and claimed herein can be made and executed without undue experimentation in light of the present disclosure.

We claim:

1. A device for articulation of the position of head and spine comprising:
a head support assembly mounted in a pitch adjustment mechanism;
a face mask;
a locking mechanism for the face mask mounted on the pitch adjustment mechanism;
the pitch adjustment mechanism attached to one end of a universal link;
an opposite end of the universal link attached to a traveling box with a roll adjustment mechanism;
the traveling box mounted on a base plate with a translation adjustment mechanism;
a couch locking mechanism attached to the base plate and designed to attach to a selected patient couch.

2. A device according to claim 1 further comprising a minimum of one sensor positioned to monitor the location of at least one adjustable mechanism.

3. A device according to claim 1 further comprising one or more sensors positioned to monitor the pitch adjustment mechanism, the roll adjustment mechanism and the translation adjustment mechanism.

4. A device according to claim 3 further comprising a minimum of one controllable movement device attached to the pitch adjustment mechanism, the roll adjustment mechanism, and the translation adjustment mechanism.

5. A device according to claim 4 wherein the one or more sensors feed into a controller for monitoring the position of an adjustment mechanism.

6. A device according to claim 5 wherein the controller controls the position of an adjustment mechanism.

7. A device for articulation of the position of head and spine comprising:
a head support assembly mounted in a pitch adjustment mechanism;
a nasal depressor assembly;
the nasal depressor assembly attached to an articulating nose depressor support assembly attached to an articulating adapter block which is attached to the pitch adjustment mechanism;
the pitch adjustment mechanism attached to one end of a universal link;
an opposite end of the universal link attached to a traveling box with a roll adjustment mechanism;
the traveling box mounted on a base plate with a translation adjustment mechanism;
a couch locking mechanism attached to the base plate and designed to attach to a selected patient couch.

8. A device according to claim 7 further comprising a minimum of one sensor positioned to monitor the location of at least one adjustable mechanism.

9. A device according to claim 7 further comprising one or more sensors positioned to monitor the pitch adjustment mechanism, the roll adjustment mechanism and the translation adjustment mechanism.

10. A device according to claim 9 further comprising a minimum of one controllable movement device attached to the pitch adjustment mechanism, the roll adjustment mechanism, and the translation adjustment mechanism.

11. A device according to claim 10 wherein the one or more sensors feed into a controller for monitoring the position of an adjustment mechanism.

12. A device according to claim 11 wherein the controller controls the position of an adjustment mechanism.
